(19) United States
(12) Patent Application Publication
(10) Pub. No.: US 2009/0012161 A9
(43) Pub. Date: Jan. 8, 2009

(54) Pharmaceutical composition comprising a beta-3-adrenoceptor agonist and a serotonin and/or norepinephrine reuptake inhibitor

(75) Inventors: Ludwig Mehlburger, Bingen (DE); Martin Michel, Amsterdam (NL); Marion Wienrich, Weiterstadt (DE); Ursula Ebinger, Boonton Township, NJ (US)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/136,165

(22) Filed: May 24, 2005

Related U.S. Application Data
(63) Continuation of application No. PCT/EP2003/012225, filed on Nov. 3, 2003.

(30) Foreign Application Priority Data
Nov. 27, 2002 (EP) 02026546

(51) Int. Cl. A61K 31/24; A61K 31/195; A61K 31/137

(52) U.S. Cl. 514/540; 514/567; 514/649

(57) ABSTRACT

This invention describes a new combination for the treatment of bladder function disorders which comprises a serotonin- and/or norepinephrine-reuptake inhibitor and a beta-3-adrenoceptor agonist.

This invention describes a new active substance combination for the treatment of functional bladder problems, particularly mixed incontinence. According to the invention a combination of pharmaceutically active substances comprising at least one beta-3-adrenoceptor agonist and at least one serotonin and/or norepinephrine reuptake inhibitor is proposed.

### PRIOR ART

The incidence of urinary incontinence is constantly increasing as a result of changes in the ageing statistics. Nevertheless those affected are for the most part still untreated or inadequately treated. Apart from the medical consequences such as chronic infections of the urinary passages, urinary incontinence for those affected is associated with a high psychological burden of suffering. It is estimated that 100 million older people are affected by urinary incontinence.

The lower urinary tract consists of the bladder, the urethra, the associated muscles and the ligaments of the suspensory apparatus. The purpose of the bladder is to store urine and evacuated it. The important factors for performing the storage function are not only the relaxation of the bladder muscle (detrusor muscle), but also the closure mechanisms provided by the neck of the bladder, the smooth muscle of the urethra and also the cross-striated muscle of the urethra and the pelvic floor. During the emptying of the bladder (micturition) the detrusor muscle contracts while the urethra and pelvic floor relax and the sphincter muscle of the bladder opens. These operations require complex control by the parasympathetic, sympathetic and somatic nervous system.

Functional bladder problems are a heterogeneous group of disorders which differ in their aetiology, diagnosis and therapy.

In the standardising recommendations of the International Continence Society (ICS) urinary incontinence is defined as involuntary loss of urine which is objectively detectable and constitutes a social and hygiene problem. Generally, urinary incontinence only occurs when there is an unintentional increase in the pressure in the bladder during the storage phase. This can happen as a result of unrestricted contractions of the detrusor muscle (urge incontinence) or failure of the urethral closure mechanism (stress incontinence).

According to the ICS definition, the term overactive bladder (OAB) is used when there is an irresistible imperative need to urinate, which may or may not be associated with urge incontinence, usually with increased frequency of micturition and nocturnal urination. Pathophysiologically, this complaint may be based on involuntary contractions during the filling phase, the cause of which may be neurogenic or non-neurogenic (idiopathic) by nature.

Urge incontinence is characterised by an irresistible need to urinate and involuntary loss of urine.

Stress incontinence is characterised by the involuntary loss of urine which generally occurs at moments of elevated intraabdominal pressure. This may occur for example when lifting, coughing, sneezing, running while at the same time there is no detrusor activity. Loss of urine takes place as the result of a variable combination of an insufficiency of the sphincter muscles of the bladder and the pelvic floor as well as anatomical defects in the suspensory apparatus. As a result the closure pressure of the urethra is too low and incontinence results. Pure stress incontinence often occurs in women, particularly if they have given birth. In men, this form of urinary incontinence is usually only observed after prostatectomies or other surgical interventions on the small pelvis.

In so-called mixed incontinence patients suffer from symptoms of both stress incontinence and urge incontinence. Once again, it is mainly women who are affected.

For the treatment of the various forms of urinary bladder functional disorders, particularly stress incontinence, urge incontinence, mixed incontinence or overactive bladder, various approaches are available.

For treating urge incontinence the WHO recommends treatment with anticholinergics (antimuscarinics). However, their use is limited because they are only moderately effective and particularly because they have serious side effects such as dryness of the mouth, accommodation disorders, constipation and central nervous effects (dizziness, fatigue, confusion).

There are, in particular, conservative and surgical options for treating stress incontinence. Hitherto, no generally applicable drug therapy has been established. Alpha-agonists such as pseudoephedrine and phenylpropanolamine show only a very modest effect in the treatment of low-grade stress incontinence. A disadvantage is that they have no selectivity for the urethral muscles and have numerous side effects such as hypertension, tachycardia, arrhythmia, sleep disorders, headaches and tremors.

The treatment of mixed incontinence is a controversial subject of discussion and encompasses combinations of invasive procedures for treating the stress incontinence component and drug therapies for treating the urge incontinence component.

Since the mid-1995s it has been reported that selective beta-3-adrenoceptor-agonists are also promising in the treatment of urinary incontinence (EP 0 958 835). As the stimulation of beta-3-receptors is of exceptional importance for the relaxation of the detrusor muscle, the use of selective beta-3-adrenoceptors in patients with urge incontinence should result in the reduction or prevention of involuntary detrusor contractions during the urine storage phase. Tests with beta-3-adrenoceptor agonists promise a high efficacy while being well tolerated. In addition, their activity should be restricted to the storage phase of the bladder and unimpeded emptying of the bladder should be guaranteed without any build-up of urine residues.

Currently, clinical trials for the treatment of stress incontinence are being conducted on those substances which selectively inhibit serotonin and/or norepinephrine reuptake from the synaptic gap into the nerve cells. These substances are known as selective serotonin/norepinephrine reuptake inhibitors and lead to a prolonging and reinforcement of the effect of serotonin (5-hydroxy-tryptamine=5-HT), and/or nor-adrenalin (NA). Through a complex mechanism of activity, selective serotonin/norepinephrine reuptake inhibitors are able to stimulate the activity of the pudendal nerve, promote the contraction of the cross-striated sphincter muscle of the bladder and thereby maintain continence. Phase III clinical trials have shown in the meantime that by using a selective serotonin/norepinephrine reuptake inhibitor it is possible for the first time to provide an effective drug therapy for stress incontinence.

There are also only limited therapies available for treating overactive bladder. The less well established forms of treatment also include drugs containing antimuscarinics as the active substance.

### PROBLEM OF THE INVENTION

Despite the many promising approaches and progress in the treatment of the various forms of urinary incontinence, which have been found to be causally complex and heterogeneous, the development of efficient and well-tolerated therapies remains a challenge.

The present invention sets out to make a contribution to the treatment of urinary incontinence in general and the various forms mentioned above. A pharmaceutical combination is provided which is intended to combine the advantages of the serotonin and/or norepinephrine reuptake inhibitors and also those of the beta-3-adrenoceptor agonists in a manner which promotes the treatment of the underlying ailment.

### DESCRIPTION OF THE INVENTION

According to the present invention a new pharmaceutical composition is provided which comprises (a) at least one serotonin and/or norepinephrine reuptake inhibitor in a pharmaceutically effective amount and (b) a beta-3-adrenoceptor-agonist in a pharmaceutically effective amount as active ingredients.

### a) ACTIVE COMPONENTS

In the description of the preferred embodiment certain terminology will be used hereinafter in the interests of clarity. This terminology should include the embodiment described and all technical equivalents which work in a similar manner for a similar purpose to achieve similar results. To the extent that any pharmaceutically active compound is disclosed or claimed, it is expressly intended that all active metabolites which are produced in vivo are included, and it is expressly intended that all enantiomers, diastereomers or tautomers are included, if the compound is capable of occurring in its enantiomeric, diastereomeric or tautomeric form. Obviously, the isomer which is pharmacologically most effective and most free from side effects is preferred. Also included are pharmacologically acceptable salts thereof. Examples of pharmaceutically active salts for each of the compounds which are the subject of this description include, without being restricted thereto, salts which are prepared from pharmaceutically acceptable acids or bases, including organic and inorganic acids and bases. If the preferred compound is basic, salts may be prepared from pharmaceutically acceptable acids. When selecting the most preferred salt, or to clarify whether a salt or the neutral compound is used, properties such as bioavailability, ease of manufacture, workability and shelf life are taken into consideration, inter alia. Suitable pharmaceutically acceptable acids include acetic acid, benzenesulphonic acid (besylate), benzoic acid, p-bromophenylsulphonic acid, camphorsulphonic acid, carbonic acid, citric acid, ethanesulphonic acid, fumaric acid, gluconic acid, glutamic acid, hydrobromic acid, hydrochloric acid, hydriodic acid, isethionic acid, lactic acid, maleic acid, malic acid, mandelic acid, methanesulphonic acid (mesylate), mucinic acid, nitric acid, oxalic acid, pamoic acid, pantothenic acid, phosphoric acid, succinic acid, sulphuric acid, tartaric acid, p-toluenesulphonic acid and the like. Examples of pharmaceutically acceptable salts include, without being restricted thereto, acetate, benzoate, hydroxybutyrate, bisulphate, bisulphite, bromide, butyn-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulphonate, methoxybenzoate, methylbenzoate, monohydrogenphosphate, naphthalene-1-sulphonate, naphthalene-2-sulphonate, oxalate, phenylbutyrate, phenylproprionate, phosphate, phthalate, phenylacetate, propanesulphonate, propiolate, propionate, pyrophosphate, pyrosulphate, sebacate, suberate, succinate, sulphate, sulphite, sulphonate, tartrate, xylenesulphonate and the like.

Insofar as it is necessary for completeness, the methods of synthesis of the compounds for which the prior art is mentioned and the dosages thereof are expressly included by reference to the prior art mentioned at the corresponding point.

The following compounds are mentioned as examples of serotonin and/or norepinephrine reuptake inhibitors, without being restricted thereto: tandamine, pirandamine, ciclazindole, fluparoxane, lortalamine, talsupram, talopram, prindamine, nomifensin, viloxazine, tomoxetine, duloxetine, venlafaxine, milnacipran, reboxetine, sibutramine, sibutramine hydrochloride, (R-) or (S-)-didesmethylsibutramine, optionally in salt form, (R-) or (S-)-desmethylsibutramine, optionally in salt form. The following may also be used: desipramine, maprotiline, nomifensin, citalopram, fluoxetine, fluvoxamine, paroxetine, sertraline, trazodone. In the English literature the compounds often have an “e” at the end of the name, e.g. duloxetine instead of the German Duloxetin.

In particularly preferred embodiments the selective serotonin and/or norepinephrine reuptake inhibitor duloxetine, N-methyl-3-(1-naphthalenyloxy)-3-(2-thienyl)propanamine and the pharmaceutically acceptable salts thereof are either in its enantiomeric (particularly (+)-enantiomeric) or racemic form and most preferably in the (+) -form.

Duloxetine, (+)-N-methyl-3-(1-naphthalenyloxy)-3-(2-thienyl)propanamine, is disclosed in U.S. Pat. No. 4,956,388 or U.S. Pat. No. 5,023,269. According to U.S. Pat. No. 5,744,474 the compound may be used to treat urinary incontinence. Duloxetine is represented by the following formula I:

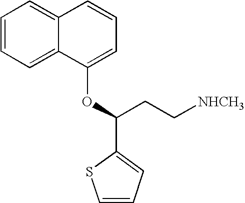

Duloxetine is preferably used in the form of the hydrochloride salt and the (+)-enantiomer.

Each of these compounds listed as serotonin/norepinephrine reuptake inhibitors may be used to treat urinary incontinence, including the sub-indications of stress incontinence, urge incontinence, mixed incontinence or hyperactive bladder as listed hereinbefore.

The second component comprises one or more beta-3-adrenoreceptor-agonists. This is preferably selected from the following group:

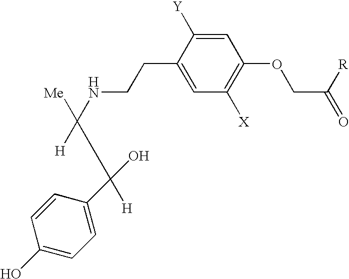

Details are disclosed in WO 00/02846 with
1) X═Br, Y═H, R═OH
2-[2-bromo-4-[2-[[(1S ,2R)-2-hydroxy-2-(4-hydroxyphenyl)-1-methylethyl]amino]ethyl]phenoxy]acetic acid,
2) X═Cl, Y═H R═OH
2-[2-chloro-4-[2-[[(1S ,2R)-2-hydroxy-2-(4-hydroxyphenyl )-1-methylethyl]amino]ethyl]phenoxy]acetic acid,
3) X═Y═Cl, R═OH

2-[2,5-dichloro-4-[2-[[(1S,2R)-2-hydroxy-2-(4-hydroxyphenyl)-1-methylethyl]amino]ethyl]phenoxy]acetic acid,
4) X═Y═H, R═OH
2-[4-[2-[[(1S,2R)-2-hydroxy-2-(4-hydroxyphenyl)-1 -methylethyl]amino]ethyl]-2,5-dimethylphenoxy]acetic acid,
5) X═OH; Y═H; R═OH
2-[2-hydroxy-4-[2-[[(1S ,2R)-2-hydroxy-2-(4-hydroxyphenyl )-1-methylethyl]amino]ethyl]phenoxy]acetic acid,
6) X═Cl;Y═H, R═OEt
ethyl-2-[2-chloro-4-[2-[[(1S ,2R)-2-hydroxy-2-(4-hydroxyphenyl)-1-methylethyl]amino]ethyl]phenoxy]acetate,
7) X═Cl; Y═Cl, R═OEt
ethyl-2-[2,5-dichloro-4-[2-[[(1S,2R)-2-hydroxy-2-(4-hydroxyphenyl)-1-methylethyl]amino]ethyl]phenoxy]acetate,
8) X=Me; Y=Me, R═OEt (-)-ethyl-2-[4-(2-{[(1S,2R)-2-hydroxy-2-(4-hydroxyphenyl)-1 -methylethyl]amino}ethyl)-2,5-dimethylphenyloxy]acetate,
9) X=Me; Y=Me, R═OH
(−)-2-[4-(2-{[(1S,2R)-2-hydroxy-2-(4-hydroxyphenyl)-1 -methylethyl]amino}ethyl-2,5-dimethylphenyloxy]acetic acid,

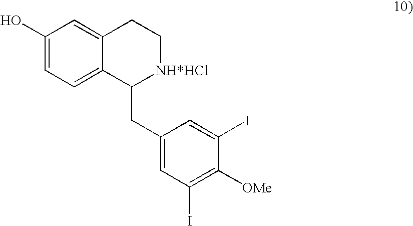

More detailed information on this substance can be found in J. Med. Chem. 44 (2001) 1456.

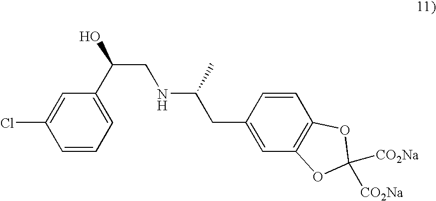

Disodium-([R,R]-5-2-[[2-(3-chlorophenyl)-2-hydroxyethyl]-amino]propyl)-1,3-benzodioxol-2,2-dicarboxylate

More detailed information on this substance can be found in J. Med. Chem. 44 (2001) 1456 or in the Journal of Urology 165 (2001) 240.

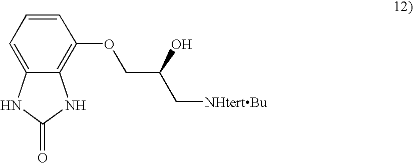

More detailed information on this substance, which is also known as CGP 12177A, can be found in the Journal of Urology 165 (2001) 240 or in J. Med. Chem. 44 (2001) 1456

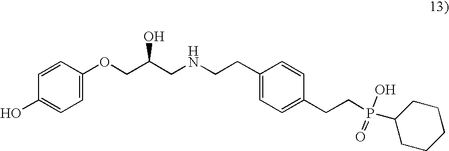

More detailed information on this substance, which is also known as SB 226552, can be found in the J. Med. Chem. 44 (2001) 1456.

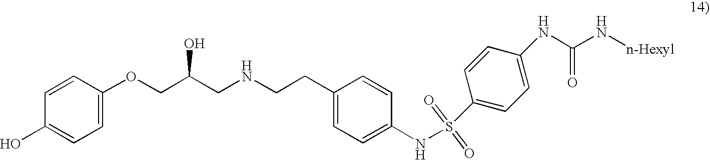

More detailed information on this substance, which is also known as L755507, can be found in J. Med. Chem. 44 (2001) 1456.

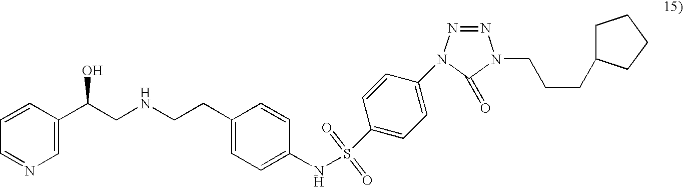

More detailed information on this substance, which is also known as L 770664, can be found in J. J. Med. Chem. 44 (2001) 1456.

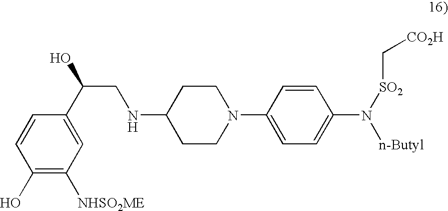

More detailed information on this substance can be found in the J. Med. Chem. 44 (2001) 1456 or in Bioorg. Med. Chem. Lett. 9 (2001) 2045. 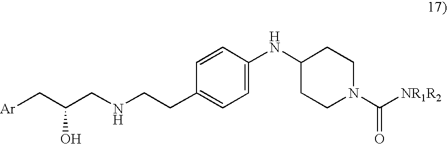
with
1) Ar=4-OHPh-O, R1=octyl, R2=H
2) Ar=4-OH,3-methylsulphonylamidophenyl-O, R1=2,5-diFbenzyl, R2=H
3) Ar=4-OH,3-methylsulphonylamidophenyl, R1=2,5-diFbenzyl, R2=H

More detailed information on these substances can be found in Bioorg. Med. Chem. Lett. 11 (2000) 3123.

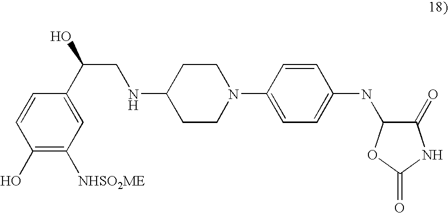

More detailed information on this substance can be found in Bioorg. Med. Chem. Let. 11 (2001) 981. 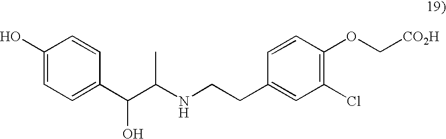
2-[2-chloro-4-(2-{[(1S, 2R)-2-hydroxy-2-(4-hydroxyphenyl)-1-methylethyl]amino}ethyl)phenoxy]acetic acid

More detailed information on this substance can be found in Med. Chem. 46 (2003) 105.

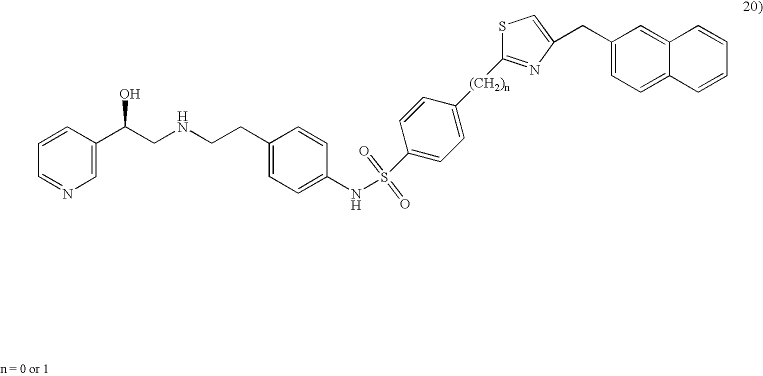

More detailed information on this substance can be found in Bioor. Med. Chem. Lett. 10 (2000) 1971.

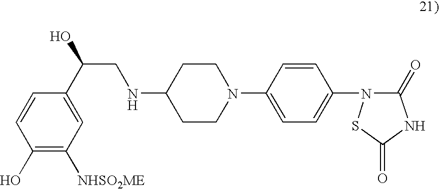

More detailed information on this substance can be found in Bioorg. Med. Chem. Lett. 11 (2001) 757.

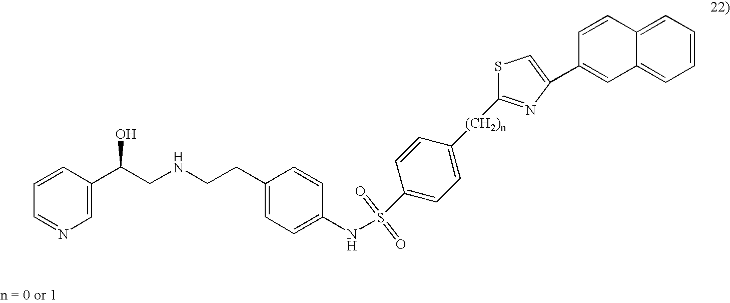

More detailed information on this substance can be found in Bioor. Med. Chem. Lett. 10(2000)1971.

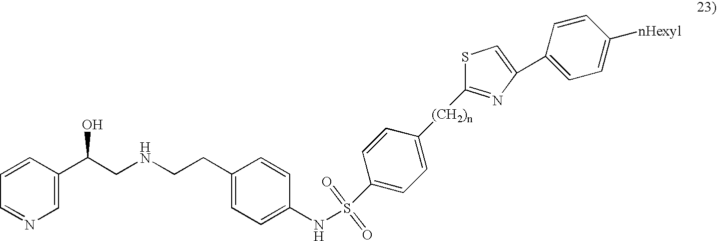

More detailed information on this substance can be found in Bioor. Med. Chem. Left. 10 (2000) 1971.

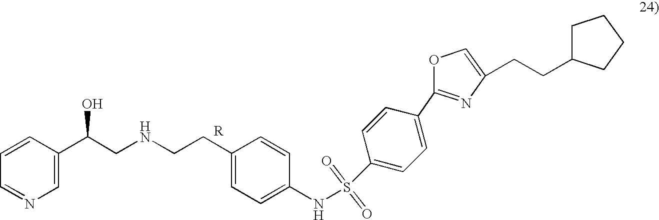

More detailed information on this substance can be found in Bioorg. Med. Chem. Lett. 10 (2000)1531.

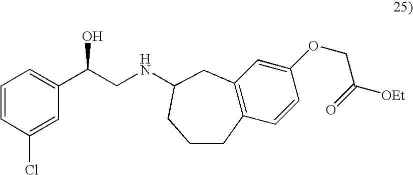

FK175
ethyl [R-(R*,S*)]- [[8-[[2-(3-chlorophenyl)-2-hydroxyethyl]amino]-6,7,8,9-tetrahydro-5H-benzocyclohepten-2-yl]oxy]-acetate, hydrochloride,

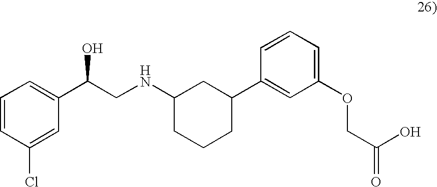

GS-332
[1S-[1α,3β(S*)]]- 3-[3-[[2-(3-chlorophenyl)-2-hydroxyethyl]amino]cyclohexyl]phenoxy]-acetic acid, monosodium salt,

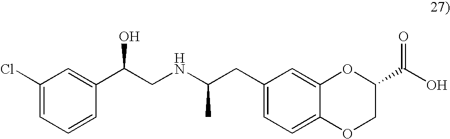

More detailed information on this compound, also known as N-5984, can be found in the literature.
28) 2-(3-{[2-(3-chlorophenyl)-2R-hydroxyl-ethylamino]ethylamino}phenyl) furan-3-carboxylic acid. Information on this compound can be found in the literature.
29) 2-(3-( [2-(3-chlorophenyl)-2R-hydroxyl-ethylamino]ethylamino}phenyl) thiophene-3-carboxylic acid. Information on this compound can be found in the literature.

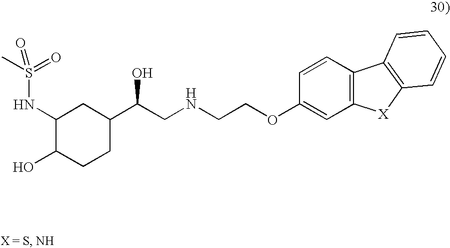

More detailed information on this compound, also known as SB-418790, can be found in the literature.

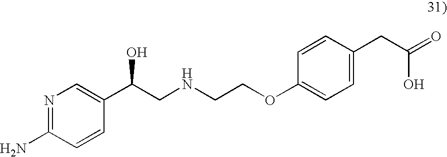

More detailed information on this compound, also known as CP-331684, can be found in the literature.

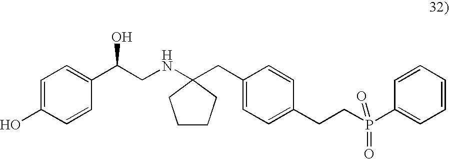

More detailed information on this compound, also known as SB-251023, can be found in the literature.

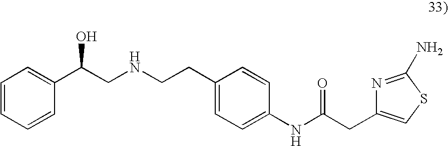

More detailed information on this compound, (R)-2-(2-aminothiazol-4-yl)-4′-[2-[2-(hydroxy-2-phenylethyl)amino]ethyl]acetanilide, can be found in the literature WO 03/037881.

According to the invention, the enantiomers, diastereomers of the above-mentioned compounds, optionally the tautomers, metabolites or pharmacologically active salts of all the abovementioned compounds are also included.

Beta-3-adrenoceptor-agonists of the catecholamine type are preferred. Most preferred are:
(−)-ethyl-2-[4-(2-{[(1S,2R)-2-hydroxy-2-(4-hydroxyphenyl)-1-methylethyl]amino}ethyl)-2,5-dimethylphenyloxy]acetate,
(−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride,
(−)-2-[4-(2-{[(1S ,2R)-2-hydroxy-2-(4-hydroxyphenyl)-1-methylethyl]amino}ethyl)- 2,5-dimethylphenyloxy]acetic acid
or other pharmacologically acceptable salts thereof.

Particularly interesting examples of beta-3-adrenoceptor-agonists are (−)-ethyl-2-[4-(2-{[(1S,2R)-2-hydroxy-2-(4-hydroxyphenyl)-1-methylethyl]amino}ethyl)-2,5dimethylphenyloxy]acetate or (−)-2-[4-(2-{[(1S ,2R)-2-hydroxy-2-(4-hydroxyphenyl)-1-methylethyl]-amino}ethyl)-2,5-dimethylphenyloxy]acetic acid, the enantiomers thereof, other enantiomers thereof and pharmacologically active salts thereof.

These compounds are disclosed in WO 00/02846 or WO 2003024916.

The last two compounds named are represented by the following formula II, which should take precedence over the preceding name in the event of any contradictions: 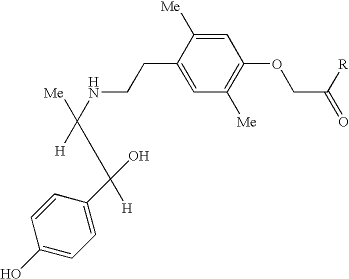
where R═Oethyl: (−)-ethyl-2-[4-(2-{[(1S,2R)-2-hydroxy-2-(4-hydroxyphenyl)-1methylethyl]amino}ethyl)-2,5-dimethylphenyloxy]acetate, preferably the monohydrate,
where R═OH: (−)-2-[4-(2-{[(1S,2R)-2-hydroxy-2-(4-hydroxyphenyl)-1-methylethyl]-amino}ethyl)-2,5-dimethylphenyloxy]acetic acid.

Particularly preferred combinations comprise a combination of (a) duloxetine either in its enantiomeric or racemic form or pharmacologically acceptable salts thereof or any active metabolites thereof and (b) at least one of the following compounds:
(−)-ethyl-2-[4-(2-{[(1S,2R)-2-hydroxy-2-(4-hydroxyphenyl)-1-methylethyl]amino}ethyl)-2,5-dimethylphenyloxy]acetate, (−)-ethyl-2-[4-(2-{[(1S,2R)-2-hydroxy-2-(4-hydroxyphenyl)-1 -methylethyl]amino}ethyl)-2,5-dimethylphenyloxy]acetate-monohydrochloride, (−)-2-[4-(2-{[(1S,2R)-2-hydroxy-2-(4-hydroxyphenyl)-1-methylethyl]amino}ethyl)-2,5-dimethylphenyloxy]acetic acid or any other pharmacologically acceptable salts thereof or any active metabolites thereof.

### b) DOSAGE

In order to determine the optimum dose of the two active substances for urinary incontinence, various basic conditions have to be taken into consideration such as for example the age and body weight of the patient, the nature and stage of the disease and the potency of the compound. This is deemed to be within the capabilities of the skilled man, and the existing literature on the components can be consulted in order to arrive at the optimum dose. The doses specified relate to the dosage after the end of the initial phase.

The doses given hereinafter expressly include all the numerical values, both whole numbers and fractions, within the range specified. The data relate to adults. Paediatric doses may be lower.

The preferred dose of the serotonin and/or norepinephrine reuptake inhibitor in humans is between 0.001 mg and 5 g per day, and is preferably between 0.001 mg and 250 mg and most preferably between 0.1 mg and 200 mg.

In some cases a smaller amount may be sufficient while in other cases a larger total amount may be required.

The total daily dose may be taken in one go or in several portions depending on the treatment plan. The treatment plan may also prescribe intervals of longer than one day between the doses.

The choice of dosage for the first component, i.e. the serotonin and/or norepinephrine reuptake inhibitor, is the dose which provides relief for the patient.

If duloxetine is used as the active substance, the daily dose desirably contains about 0.1 mg to about 500 mg. More preferably each dose of the component contains about 0.5 to about 160 mg of the active substance. This dosage form enables the full daily dose to be taken in half or whole, single or repeated doses. Doses taken more than once a day or twice a day (e.g. 3, 4, 5 or 6 doses per day) are also expressly included.

The average daily dose for adults of the other norepinephrine reuptake inhibitors is as follows. The doses expressly include all the numerical values, both whole numbers and fractions, within the range specified. Paediatric doses may be lower.

Average daily dose of the component (mg/day/patient)

tandamine 7.5 to 3750, pirandamine 7.5 to 3750, ciclazindole 5 to 500, fluparoxan 75 to 750, lortalamine 1 to 200, talsupram 1 to 3750, talopram 1 to 3750, prindamine 1 to 3750, nomifensin 1 to 80, viloxazine 1 to 3750, tomoxetine 1 to 200, venlafaxin 2 to 200, milnacipran 7.5 to 75.

As is well known, the doses and the treatment plan (i.e. one, two, three or more doses per day) of the second component depend on the factors to which reference was made in conjunction with the choice of dosage for the first component.

The average daily dose for adults of the second component (beta-3-agonist) is about 10 mg to about 750 mg per day, preferably 5 to 120 mg, more preferably 10 to 100 mg, administered in one or more doses. The doses expressly include all the numerical values, both whole numbers or fractions, within the range specified.

For both components the paediatric doses may be lower.

### c) FORMULATIONS

The compositions of the present invention may conveniently be administered in a pharmaceutical composition which contains the active components in combination with a suitable carrier. Such pharmaceutical compositions may be prepared by methods and contain carriers which are well known in the art. Generally recognised textbooks are available to the skilled man for this purpose.

The compositions of the present invention may be administered parenterally (e.g. by intravenous, intraperitoneal, subcutaneous or intramuscular injection), topically, orally, intranasally, intravaginally, transdermally, rectally, by pulmonary or nasal inhalation, oral administration being particularly preferred. Of the oral formulations, those which are resistant to gastric juices are preferred. Therefore capsules or tablets resistant to gastric juices are preferred, and in both cases may be made with a coating which is resistant to gastric juices. It is particularly preferred that the serotonin/norepinephrine reuptake inhibitor be formulated in a preparation which is resistant to gastric juices. The skilled man will find instructions for formulations resistant to gastric juices in the prior art.

Various formulating options are described below. The skilled man may choose a suitable formulation from them.

For oral therapeutic administration the composition according to the invention may be combined with one or more carriers and used in the form of tablets for swallowing, buccal tablets, sublingual tablets, sugar-coated tablets, sprays, powders, pastilles, coated tablets, granules, capsules, elixirs, suspensions, solutions, syrups, lozenges, chewing gums, foods and the like.

A spray may be prepared for example by grinding the particles of active substance to a suitable size.

Dilute sprays may be prepared by finely grinding the powdered substance with a non-toxic carrier material such as lactose and delivering it as a spray. Other suitable carrier materials for this purpose are other carbohydrates such as starch or mannitol. These sprays may optionally contain flavourings, preservatives, dispersing agents, colourings and other pharmacological adjuvants.

Capsules may be prepared from a powder of the kind described above or other powders, which are placed in a capsule, preferably a gelatine capsule, and the capsule is then sealed.

It is also possible for lubricants known from the prior art to be introduced into the capsule or used to seal the two parts of the capsule. The efficacy of a capsule when taken orally can be increased by the addition of disintegrating or solubilising substances such as, for example, carboxymethylcellulose, carboxymethylcellulose calcium, low-substituted hydroxypropylcellulose, calcium carbonate, sodium carbonate and other substances. The active substance may be present in the capsule not only as a solid but also in suspended form, for example in vegetable oil, polyethyleneglycol or glycerol using surface-active substances, etc.

Tablets may be prepared by compressing the powdered mixture and then processing it into granules, for example. The tablets may contain various excipients such as e.g. starches, lactose, sucrose, glucose (e.g. for vaginal tablets), sodium chloride, urea for tablets for dissolving or injecting, amylose, various types of cellulose as described above and others. Glycerol or starch may be used as a moisture retaining agent.

The disintegrants used may be, for example, starch, alginic acid, calcium alginate, pectic acid, powdered agar-agar, formaldehyde gelatine, calcium carbonate, sodium bicarbonate, magnesium peroxide and amylose.

Anti-disintegrants or solution retardants which may be used include, for example, sucrose, stearin, solid paraffin (preferably with a melting point in the range from 50-52° C.), cocoa butter and hydrogenated fats.

Other disintegrants may be: corn starch, potato starch, alginic acid and the like.

Suitable absorption accelerators include, inter alia, quaternary ammonium compounds, sodium lauryl sulphate and saponins.

Ether may be used, for example, as a binder distributor and cetyl alcohol, glycerol monostearate, starch, corn starch, lactose, wetting agents (e.g. aerosol OT, Pluronics, Tweens), gum tragacanth, gum arabic, gelatine and others may be used as hydrophilising agents or disintegration accelerators.

Sucrose, fructose, lactose or aspartame may be used as sweeteners while peppermint, wintergreen oil, cherry flavouring etc may be used as flavouring agents.

The following may also be generally used as additional excipients: Aerosil, Aerosol OT ethylcellulose, Amberlite resin, XE-88, Amijel, Amisterol, amylose, Avicel microcrystalline-cellulose, bentonite, calcium sulphate, Carbowax 4000 and 6000, carrageenan, castor wax, cellulose, microcrystalline cellulose, crospovidone, dextran, dextrin, dicalcium phosphate, pharmaceutical tablet base, kaolin, lactose (USP), lactosil, magnesium stearate, mannitol, granular mannitol N. F. methylcellulose, Miglyol 812 neutral oil, powdered milk, powdered sugar, nal-tab, nepol-amylose, Pöfizer crystalline sorbitol, plasdone, polyethyleneglycols, polyvinyl acetate phthalate, polyvinylpyrrolidone, Precirol, neat's foot oil (hydrogenated), melting tablet base, silicone, stabiline, Sta-rx 1500, syloid, Waldhof tablet base, tablettol, talcum cetylatum and stearatum, Tego metal soaps, fructose and tylose. The tabletting excipient K (M25) is particularly suitable, and also complies with the requirements of the following pharmacopoeias: DAB, Ph, Eur, BP and NF.

Other excipients which may be used can be found in the examples, but other excipients known from the prior art may also be used.

The tablets may be produced by direct compression, for example.

It is also possible to prepare other formulations for oral administration such as solutions, syrups, elixirs etc. If desired the compound may be micro-encapsulated.

Parenteral administration may be achieved by dissolving the compound in a liquid and injecting it by subcutaneous, intramuscular or intravenous route. Suitable solvents include, for example, water or oily media.

In order to prepare suppositories the compound may be formulated with low-melting and water-soluble or water-insoluble materials such as polyethylene glycol, cocoa butter, higher esters (for example moerysthyl, palmitate) or mixtures thereof.

The above list is provided by way of example and a skilled man might consider other excipients.

Various other materials may be provided as coatings or for modifying the physical form of the solid dosage units in some other way. For example, tablets, pills or capsules may be coated with gelatine, wax, shellac or sugar and the like. As already mentioned, formulations resistant to gastric juices are preferred for the oral preparations. Therefore, gastric juice-resistant coatings are preferred for tablets or capsules. In the case of a syrup or elixir, sucrose or fructose may be used as the sweetener, methyl- and propylparaben may be present as preservatives and a colouring and a flavouring agent such as cherry or orange flavour may also be present.

The excipients mentioned above are not restricted to the use of the formulation in connection with which they have been mentioned but may also be applied to the other formulations.

Naturally, any material used in the preparation of any of these dosage units must be pharmaceutically acceptable and substantially non-toxic in the amounts used. In addition, the active components may be incorporated in preparations with delayed release and devices which, without being restricted thereto, include those based on osmotic pressures, in order to achieve the desired release profile. One-a-day formulations for each of the active components are particularly included.

Compositions and preparations of this kind should contain at least 0.1% of active compound. The percentage of the compositions and preparations may naturally vary and may appropriately make up between 0.1 and about 100% of the weight of a given dosage unit. The quantity of active compound in therapeutically useful compositions of this kind is such that an effective dose is present.

The composition according to the invention which contains the two active components may be administered in the same physical form or at the same time in accordance with the dosages described above and in the administration carriers described above. The dosages for each active component may be measured separately and may be administered as a single combined dose or separately. They may be given at the same time or at different times provided that both active ingredients come to act in the patient at some time over a 24 hour period. It is preferable if the two components act in such a way as to achieve an effect which is better than the individual activity in each case. Simultaneous or coincident administration means that the patient takes one drug within about five minutes of taking the other drug. For ease of handling it is preferable to use formulations in which the two drugs are given to the patient close together and typically at the same time. Then the actual time of administering each drug might not be so important.

### d) INDICATIONS

The pharmaceutical composition according to the invention may preferably be used to treat or prevent, inter alia, each of the syndromes mentioned below, as an individual syndrome and in conjunction with another of the syndromes mentioned, without being restricted thereto. Urinary incontinence, particularly stress incontinence, urge incontinence, mixed incontinence or overactive bladder of neurogenic or non-neurogenic origin and further sub-indications thereof.

Thus, the invention includes both those syndromes whose cause is dysfunction or disease of an organ and those which can be attributed to diseases or disorders of the central nervous system. Accordingly, every treatment of bladder function disorder, particularly urinary incontinence of all kinds, is taken into account by the present invention.

Thus, a further embodiment of the present invention comprises using the composition according to the invention to prepare a drug for treating or preventing any of the indications of bladder dysfunction mentioned in the preceding paragraph.

The above diseases or disorders are treated by administering a therapeutically effective amount of the composition according to the invention to a mammal. In most cases this is a human being but the treatment of farm animals (e.g. cattle) and domestic animals (e.g. dogs, cats and horses) is also expressly covered. For use in veterinary medicine the dosages used may be different from those specified herein.

It is expected that the new composition will provide rapid relief for those suffering from the above diseases and disorders with a minimum amount of harmful side effects.

### e) EXAMPLES

The invention is illustrated by the following non-restrictive Examples.

Particularly preferred combinations are
duloxetine and (−)-ethyl-2-[4-(2-{[(1S ,2R)-2-hydroxy-2-(4-hydroxyphenyl)-1 -methyl-ethyl]amino}ethyl)-2,5-dimethylphenyloxy]acetate.
duloxetine and (−)-ethyl-2-[4-(2-{[(1S ,2 R)-2-hydroxy-2-(4-hydroxyphenyl )-1ethyl]amino}ethyl)-2,5-dimethylphenyloxy]acetate-monohydrochloride.
duloxetine and (−)-2-[4-(2-{[(1S,2R)-2-hydroxy-2-(4-hydroxyphenyl)-1 -methylethyl]-amino}ethyl)-2,5-dimethylphenyloxy]acetic acid.
duloxetine and (−)-2-[4-(2-{[(1S,2R)-2-hydroxy-2-(4-hydroxyphenyl)-1-methylethyl]-amino}ethyl)-2,5-dimethylphenyloxy]acetic acid-monohydrochloride.

After the invention has been described in detail and with reference to the preferred embodiments it is clear that modifications and adaptations are possible without deviating from the scope of the accompanying claims.

### Example N^{o} 1

### Composition comprising (−)-ethyl-2-[4-(2-{[(1S,2R)-2-hydroxy-2-(4-hydroxyphenyl)-1-methylethyl]amino}ethyl)-2,5-dimethylphenyloxy]acetate-monohydrochloride and duloxetine: Film-Coated Tablet 20 mg/20 mg

| | |
|---|---|
| Ingredients | mg/tablet |

Core

| | |
|---|---|
| (−)-ethyl-2-[4-(2-{[(1S,2R)-2-hydroxy-2-(4- | 21.820 |
| hydroxyphenyl)-1-methylethyl]amino}ethyl)- | |
| 2,5-dimethylphenyloxy]acetate- | |
| monohydrochloride | |
| duloxetine hydrochloride | 22.460 |
| lactose monohydrate | 78.120 |
| Avicel (PH 101) | 45.000 |
| maize starch | 6.300 |
| purified water | (q.s.) |
| sodium starch glycolate | 3.600 |
| magnesium stearate | 1.800 |
| highly dispersed silicon dioxide | 0.900 |

Interlayer

| | |
|---|---|
| hydroxypropylmethylcellulose 2910 | 2.750 |
| polyethyleneglycol 400 | 0.325 |
| titanium dioxide | 1.000 |
| talc | 0.925 |
| purified water | (q.s.) |

Gastric juice-resistant coating

| | |
|---|---|
| Sureteric (polyvinyl acetate phthalate) | 14.950 |
| silicone emulsion | 0.050 |
| purified water | (q.s.) |
| total weight of tablet | 200.000 |

### Example N^{o} 2

### Composition comprising (−)-ethyl-2-[4-(2-{[(1S,2R)-2-hydroxy-2-(4-hydroxyphenyl)-1 -methylethyl]amino}ethyl)-2,5-dimethylphenyloxy]acetate-monohydrochloride and duloxetine: Film-Coated Tablet 80 mg/60 mg

| | |
|---|---|
| Ingredients | mg/tablet |

Core

| | |
|---|---|
| (−)-ethyl-2-[4-(2-{[(1S,2R)-2-hydroxy-2-(4- | 87.280 |
| hydroxyphenyl)-1-methylethyl]amino}ethyl)- | |
| 2,5-dimethylphenyloxy]acetate- | |
| monohydrochloride | |
| duloxetine hydrochloride | 67.380 |
| lactose monohydrate | 185.340 |
| Avicel (PH 101) | 125.000 |
| hydroxypropylmethylcellulose | 10.000 |
| purified water | (q.s.) |
| crospovidone | 10.000 |
| magnesium stearate | 10.000 |
| highly dispersed silicon dioxide | 5.000 |

Gastric juice-resistant coating

| | |
|---|---|
| Eudragit L 30 D-55 (copolymer of methacrylic | 136.000⁺ |
| acid and ethyl acrylate) | |
| triethyl citrate | 4.000 |
| Polysorbate 80 | 4.000 |
| glycerol monostearate | 1.200 |
| purified water | (q.s.) |
| total weight of tablet | 550.000 |

⁺Eudragit L 30 D-55 is a 30% suspension. The water content is removed during spraying.

### Example N^{o} 3

### Composition comprising (−)-ethyl-2-[4-(2-{[(1S,2R)-2-hydroxy-2-(4-hydroxyphenyl)-1-methylethyl]amino}ethyl )-2,5-dimethylphenyloxy]acetate-monohydrochloride and duloxetine: hard gelatine capsule filled with pellets 20 mg/20 mg

Duloxetine pellets

| Ingredients | mg/tablet |
|---|---|
| duloxetine hydrochloride | 22.460 |
| microcrystalline cellulose pellets | 60.000 |
| β-lactose | 80.040 |
| hydroxypropylcellulose | 1.500 |
| crospovidone | 6.000 |
| purified water | (q.s.) |

Interlayer

| Ingredients | mg/tablet |
|---|---|
| hydroxypropylmethylcellulose | 3.400 |
| purified water | (q.s.) |

Gastric juice-resistant coating

| Ingredients | mg/tablet |
|---|---|
| HPMCAS-LF | 20.400 |
| triethylcitrate | 4.080 |
| talc | 2.120 |
| purified water | (q.s.)* |

### (−)-ethyl-2-[4-(2-[(1S,2R)-2-hydroxy-2-(4-hydroxyphenyl)-1-methyl -ethyl]amino}ethyl)-2,5-dimethylphenyloxy]acetate-monohydrochloride pellets

| Ingredients | mg/tablet |
|---|---|
| (−)-ethyl-2-[4-(2-{[(1S,2R)-2-hydroxy-2-(4- | 21.820 |
| hydroxyphenyl)-1-methylethyl]amino}ethyl)- | |
| 2,5-dimethylphenyloxy]acetate- | |
| monohydrochloride | |
| microcrystalline cellulose pellets | 60.000 |
| hydroxypropylcellulose | 0.800 |
| talc | 2.380 |
| purified water | (q.s.) |

| | |
|---|---|
| Ingredients | mg/tablet |

Film coating

| | |
|---|---|
| hydroxypropylmethylcellulose | 4.250 |
| purified water | (q.s.) |

Capsule

| | |
|---|---|
| duloxetine pellets with gastric juice-resistant | 200.000 |
| coating | |
| (-)-ethyl-2-[4-(2-{[(1S,2R)-2-hydroxy-2-(4- | 89.250 |
| hydroxyphenyl)-1-methylethyl]amino}ethyl)- | |
| 2,5-dimethylphenyloxy]acetate- | |
| monohydrochloride pellets with film coating | |
| hard gelatine capsule (size 0) | 98.000 |
| total weight of capsule | 387.250 |

What is claimed is:

1. A pharmaceutical composition comprising as active components: (a) a pharmaceutically effective amount of one or more serotonin-or norepinephrine-reuptake inhibitors or both, and (b) a pharmaceutically effective amount of one or more beta-3-adrenoceptor agonists.

2. The pharmaceutical composition according to claim 1, wherein the serotonin-or norepinephrine-uptake inhibitor is selected from among tandamine, pirandamine, ciclazindole, fluparoxane, lortalamine, talsupram, talopram, prindamine, nomifensin, viloxazin, tomoxetin, duloxetine, venlafaxin, milnaxipran, reboxetin and mixtures thereof.

3. The pharmaceutical composition according to claim 1, wherein the beta-3-adrenoceptor agonist is (−)-ethyl-2-[4-(2-{[(1S ,2R)-2-hydroxy-2-(4-hydroxyphenyl)-1-methylethyl]-amino}-ethyl)-2,5-dimethylphenyloxy]acetate or (−)-2-[4-(2-([(1S,2R)-2-hydroxy-2-(4-hydroxyphenyl)-1-methylethyl]amino}ethyl)-2,5-dimethylphenyloxy] acetic acid, or a pharmacologically acceptable salt thereof or an enantiomer thereof.

4. The pharmaceutical composition according to claim 1, wherein the serotonin or norepinephrien uptake inhibitor is duloxetine which is present in either its racemic or (+)-enantiomeric form and the beta-3-adrenoceptor agonist is (−)-ethyl-2-[4-(2-{[(1S,2R)-2-hydroxy-2-(4-hydroxyphenyl)-1-methylethyl]-amino}-ethyl)-2,5-dimethylphenyloxy] acetate or (−)-2-[4-(2-{[(1S,2R)-2-hydroxy-2-(4-hydroxyphenyl )-1-methylethyl]amino}ethyl)-2,5-dimethyl phenyloxy] acetic acid, or a pharmaceutically acceptable salt thereof.

5. The pharmaceutical composition according to claim 4 which contains about 0.1 mg to about 500 mg of duloxetine and about 10 mg to about 750 mg of the beta-3-adrenoceptor agonist

6. The pharmaceutical composition according to claim 5 for rectal, vaginal, topical, oral, sublingual, intranasal, transdermal or parenteral administration.

7. The pharmaceutical composition according to claim 5 which will administer the two active components (a) and (b) simultaneously.

8. The pharmaceutical composition according to claim 5, wherein at least one of the two active components is at least partially released after some delay.

9. The pharmaceutical composition according to claim 5, wherein at least one of the two active components is at least partially released immediately.

10. A method of treating bladder function disorders such as urinary incontinence, particularly stress incontinence, urge incontinence, mixed incontinence, hyperactive bladder, or a corresponding subindication or a disease or disorder of the central nervous system which is connected to bladder function, in a mammal, which comprises administering a composition according to claim 1 to the mammal.

11. The method according to claim 10, wherein the bladder function disorder is selected from among urinary incontinence, urge incontinence, stress incontinence, mixed incontinence, other forms of urinary incontinence and hyperactive bladder.
